(19) United States
(12) Patent Application Publication
(10) Pub. No.: US 2023/0201376 A1
(43) Pub. Date: Jun. 29, 2023

(54) NEUROGENIC TISSUE NANOTRANSFECTION IN THE MANAGEMENT OF CUTANEOUS DIABETIC POLYNEUROPATHY

(71) Applicant: THE TRUSTEES OF INDIANA UNIVERSITY, Bloomington, IN (US)

(72) Inventors: Chandan K. SEN, Indianapolis, IN (US); Subhadip GHATAK, Indianapolis, IN (US); Sashwati ROY, Indianapolis, IN (US); Savita KHANNA, Indianapolis, IN (US)

(21) Appl. No.: 17/921,242

(22) PCT Filed: Apr. 29, 2021
(86) PCT No.: PCT/US2021/029780

§ 371 Date: Oct. 25, 2022

Related U.S. Application Data
(60) Provisional application No. 63/018,900, filed on May 1, 2020.

(51) Int. Cl. A61K 48/00; A61K 38/17; A61P 25/02

(52) U.S. Cl. CPC A61K 48/0075; A61K 38/1709; A61P 25/02

(57) ABSTRACT

Compositions and methods are provided for reprogramming demal fibroblasts to exhibit neurogenic properties including increased cell expression of NGF and Nt3 in vivo. In accordance with one embodiment such compositions are used in conjunction with standard treatment for use in treating neuropathic pain and stabilizing or stimulating production of PGP9.5+ mature nerve fiber in a diabetic patient’s tissues.

### CROSS REFERENCE TO RELATED APPLICATIONS

This application claims priority to the following: U.S. Provisional Pat. Application No. 63/018,900 filed on May 1, 2020, the disclosure of which is expressly incorporated herein.

### STATEMENT OF US GOVERNMENT SUPPORT

This invention was made with government support under DK114718, GM108014, NR015676, NS042617, and NS085272 awarded by National Institutes of Health. The government has certain rights in the invention.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 4 kilobytes ACII (Text) file named “335002_ST25.txt,” created on Apr. 21, 2021.

### BACKGROUND OF THE DISCLOSURE

Diabetic neuropathy is a serious diabetes complication that may affect as many as 50% of people with diabetes. High blood sugar (glucose) can injure nerves throughout the body, and most often damages nerves in an individual’s legs and feet. However, depending on the affected nerves, diabetic neuropathy symptoms can range from pain and numbness in the legs and feet to problems with the digestive system, urinary tract, blood vessels and heart.

Prior approaches to support nerve fibers in patients experiencing diabetic peripheral neuropathy (DPN) relied on pharmacologic therapies for correcting intracellular signaling pathways, biochemistry, and organelle functions of the neurons. However, such interventions have failed to proceed past phase II or III clinical trials mainly for lack of efficacy and/or adverse side-effects. Nerve growth factor (NGF) is abundantly produced by keratinocytes and is depleted at the onset of DPN. Withdrawal of NGF in vitro leads to distal axonal degeneration. Neurotrophic factor supplement, namely exogenous NGF injection, has been tested clinically as a prophylactic measure in DPN. However, therapeutic administration of exogenous NGF alone has not proceeded through clinical trials because of barriers such as injection site pain, questionable efficacy, and potential need for other trophic factors to be co-administered.

In addition to strategies that attempt to maintain healthy blood glucose levels in diabetics, there remains a need for more effective treatment strategies to prevent diabetic neuropathy, alleviate symptoms associated with DPN, or slow its progress.

### SUMMARY

In accordance with one embodiment of the present disclosure, compositions and methods are provided for preventing or treating diabetic neuropathy. Recently, a novel non-viral tissue nanotransfection technology (TNT) has been reported for in vivo reprogramming of the skin. Applicant discovered that TNT delivery of nucleic acid sequences encoding Achaete-Scute Family BHLH Transcription Factor 1 (ASCL1), Master Neural Transcription Factor BRN2 (encoded by POU3F2), and Myelin Transcription Factor 1 Like (MYT1L), achieved direct conversion of skin fibroblasts to mature electrophysiologically active induced neuronal (iN) cells (D. Gallego-Perez et al, Nat Nanotechnol. (2017)12:974-979). However, while that discovery was focused on the reprogrammed cells, applicant has now discovered that the neurotrophic environment generated in response to in vivo reprogramming of skin fibroblasts produces a neurotrophic milieu of the skin that can be leveraged to rescue and/or protect pre-existing nerve fibers that are vulnerable to degeneration under chronic diabetic conditions.

In accordance with one embodiment of the present disclosure, compositions and methods are provided for increasing the skin stroma cell expression of NGF and Nt3 expression in vivo. In one embodiment the method comprises the step of introducing nucleic acid sequences that encode for Ascl1, Brn2, and Myt1l into said patient’s skin stroma cells via tissue nanotransfection.

In accordance with one embodiment of the present disclosure, compositions and methods are provided for reprogramming human dermal fibroblasts to be induced neuronal (iN) cells wherein the reprogrammed dermal fibroblasts have the capacity to enhance the local the neurotrophic environment and alleviate symptoms associated with diabetic neuropathy. In one embodiment the human dermal cells to be reprogrammed are located in a localized region of tissue that is the source of neuropathic pain. In one embodiment the human dermal cells are reprogrammed by transfection with one or more nucleic acid sequences encoding for one or more gene products selected from Ascl1, Brn2, and Myt1l. As described herein the combined administration of Ascl1, Brn2, and Myt1l is referred to herein as “ABM” administration).

In one embodiment tissue nanotransfection (TNT) is used to deliver Ascl1, Brn2, and Myt1l (TNTABM) to directly convert skin fibroblasts into electrophysiologically active induced neuronal cells (iN) in vivo. In addition neurogenic conversion of skin fibroblasts cells, TNTABM also causes neurotrophic enrichment of the skin stroma and such enrichment of the neurotrophic milieu of the skin can rescue stressed pre-existing nerve fibers that are present under chronic diabetic conditions. Topical cutaneous TNTABM causes elevation of endogenous NGF and other co-regulated neurotrophic factors such as Nt3.

In accordance with one embodiment a method of treating neuropathy in a patient is provided. The method comprises reprogramming human dermal fibroblast cells in vivo in localized tissues experiencing weakness, numbness, and pain from nerve damage, including localized tissues associated with the hands and feet of a subject. In one embodiment the human dermal fibroblasts of the localized tissue are reprogrammed by introducing into the cytosol of the target human dermal fibroblast cells nucleic acid sequences that encoding for Ascl1, Brn2, and Myt1l, wherein the resulting reprogrammed human dermal fibroblast cells exhibit one or more neurogenic properties relative to the original fibroblast cell, including one or more of the following properties: 1) enhanced Ngf expression; 2) enhanced expression of the neurotrophic factor gene Nt3; and increased numbers of TuJ1+ cells associating with said reprogrammed cells in the dermis. In one embodiment the reprogrammed human dermal fibroblast cells continue to express fibroblast specific protein-1 (Fsp-1). In one embodiment the method is used to treat peripheral neuropathy in a diabetic patient. In one embodiment the transfection of human dermal fibroblast cells is conducted using in vivo tissue nanotransfection.

In one embodiment a method of stabilizing or stimulating PGP9.5+ mature nerve fiber in a diabetic patient’s tissues is provided. The method comprises the step of reprogramming dermal fibroblasts in vivo to become neurogenic by introducing nucleic acid sequences that encode for Ascl1, Brn2, and Myt1l into said patient’s dermal fibroblast cells, to produce reprogrammed dermal fibroblasts. In one embodiment localized dermal fibroblasts of peripheral tissues such as the hands and feet are transfected with nucleic acid sequences that encode for Ascl1, Brn2, and Myt1l, optionally via tissue nanotransfection to provide morphofunctional restoration of peripheral nerves.

### BRIEF DESCRIPTION OF THE DRAWINGS

FIGS. 1A-1I: Delivery of genes encoding Ascl1, Brn2 and Myt1l (ABM) via nanochannel electroporation (NEPABM) transfection induced neurotrophic factors in Mouse Embryonic Cells (MEF) cells. Delivery of Ascl1, Brn2 and Myt1l in MEF cells by nanochannel electroporation was confirmed via immunostaining. Phenotypic characterization revealed induced neuron-like cells 2 weeks post-NEP or 4 weeks post-NEP. Ngf expression at week 1 (FIG. 1A) and week 4 (FIG. 1B) post-NEP show elevated Ngf at 4 weeks. FIG. 1C provides a quantification of the results of an NGF ELISA from differentiated MEF media at 4 weeks post-NEP (n=10). Data from RT-qPCR analysis of mRNA for brain-derived neurotrophic factor (Bdnf; FIG. 1D and 1E), neurotrophin-3 (Nt3; FIGS. 1F and 1G), and neurotrophin-4/5 (Nt4/5; FIGS. 1H and 1I) are presented for cells at 1 week (FIGS. 1D, 1F and 1H; n=4) and at 4 weeks (FIGS. 1E, 1G and 1I; n=6) post-NEP. Neurotrophin-3 (Nt3) showed significant increase in the expression at 4 weeks post-NEPABM. Data expressed as mean ± SEM, *p < 0.05.

FIGS. 2A-2C: Tissue nanotransfection (TNT) delivery of Ascl1, Brn2, and Myt1l (TNTABM) into the dorsal skin of C57B1/6 mice results in stromal reprogramming. Confocal microscopic images showed three-plex in situ hybridization of Ascl1, Brn2, Myt1l, counterstained with DAPI. A graph representing the data generated from RT-qPCR analysis of ABM gene expression in skin 24 h post-TNT is presented (FIG. 2A; n = 4). Immunostaining showed TuJ1 fibers in skin, and quantification of TuJ1+ fiber length per mm epidermis length is provided in the bar graph of FIG. 2B (n = 6). Confocal microscopic images of skin showed co-localization of FSP and TuJ1, and quantification of TuJ1 and FSP positive cells per field of view is provided in FIG. 2C. Data expressed as mean ± SEM (n = 3-4), *p < 0.05.

FIGS. 3A-3F: TNTABM increased neurotrophic factor in skin of C57B1/6 mice. Bar graphs of data from RT-qPCR analysis of Ngf (FIG. 3A; n = 6), NGF expression quantified by ELISA (FIG. 3B; n = 8), *p < 0.01, and quantification of confocal microscopic images showing NGF in epidermis (FIG. 3C; n = 4) are presented. Bar graphs of data from RT-qPCR analysis of Bdnf, Nt3 or Nt4/Nt5 expression in skin is presented in FIGS. 3D, 3E and 3F, respectively. Data expressed as mean ± SEM (n = 6), *p < 0.05.

FIGS. 4A-4E: TNTABM increased NGF production and PGP9.5+ nerve fibers in skin of db/db mice. Quantitation of TuJ1+ fiber length per mm in immunostained TuJ1+ fibers in skin epidermis is provided in FIG. 4A (n = 6). Tissue NGF was quantified by ELISA and the data is presented in FIG. 4B (n = 9.10), *p < 0.01. Quantification of the IHC images at 4 weeks (FIG. 4C) and at 9 weeks (FIG. 4D) is provided. Quantification of the number of PGP9.5+ fibers per mm epidermis length based on immunostaining of NGF in epidermis at 9 weeks (FIG. 4E) post-TNTABM is provided. Immunostaining indicated increased number of PGP9.5+ fibers in skin. Data are mean ±SE (n = 4), *p < 0.01.

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

Tissue nanotransfection (TNT) is an electroporation-based technique capable of delivering nucleic acid sequences and proteins into the cytosol of cells at nanoscale. More particularly, TNT uses a highly intense and focused electric field through arrayed nanochannels, which benignly nanoporates the juxtaposing tissue cell members, and electrophoretically drives cargo (e.g., nucleic acids or proteins) into the cells.

As used herein a “control element” or “regulatory sequence” are non-translated regions of a functional gene, including enhancers, promoters, 5′ and 3′ untranslated regions, which interact with host cellular proteins to carry out transcription and translation. Such elements may vary in their strength and specificity. “Eukaryotic regulatory sequences” are non-translated regions of a functional gene, including enhancers, promoters, 5′ and 3′ untranslated regions, which interact with host cellular proteins of a eukaryotic cell to carry out transcription and translation in a eukaryotic cell including mammalian cells.

As used herein a “promoter” is a sequence or sequences of DNA that function when in a relatively fixed location in regard to the transcription start site of a gene. A “promoter” contains core elements required for basic interaction of RNA polymerase and transcription factors and can contain upstream elements and response elements.

As used herein an “enhancer” is a sequence of DNA that functions independent of distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. As used herein an exogenous sequence in reference to a cell is a sequence that has been introduced into the cell from a source external to the cell.

As used herein the term “non-coded (non-canonical) amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5X SSC (150 mM NaCI, 15 mM trisodium citrate), 50 mM sodium phosphate (pH 7.6), 5X Denhardt’s solution, 10% dextran sulfate, and 20 ug/ml denatured, sheared carrier DNA such as salmon sperm DNA, followed by washing the hybridization support in 0.1 X SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “phosphate buffered saline” or “PBS” refers to aqueous solution comprising sodium chloride and sodium phosphate. Different formulations of PBS are known to those skilled in the art but for purposes of this invention the phrase “standard PBS” refers to a solution having have a final concentration of 137 mM NaCl, 10 mM Phosphate, 2.7 mM KCl, and a pH of 7.2-7.4.

As used herein, the term “treating” includes alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein an amino acid “substitution” refers to the replacement of one amino acid residue by a different amino acid residue.

As used herein, the term “conservative amino acid substitution” is defined herein as exchanges within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys; Ornithine (Orn)
IV. Large, aliphatic, nonpolar residues:
   Met, Leu, Ile, Val, Cys, Norleucine (Nle), homocysteine (hCys)
V. Large, aromatic residues:
   Phe, Tyr, Trp, acetyl phenylalanine, napthylalanine (Nal)

As used herein the term “patient” without further designation is intended to encompass any warm blooded vertebrate domesticated animal (including for example, but not limited to livestock, horses, cats, dogs and other pets) or human that receives a medication or medical procedure either with or without supervision by a physician.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini.

The term “amino acid sequence” refers to a series of two or more amino acids linked together via peptide bonds wherein the order of the amino acids linkages is designated by a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M , methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g. , BrdU), and non-phosphodiester internucleoside linkages (e.g. , peptide nucleic acid (PNA) or thiodiester linkages) . In particular, nucleic acids can include, without limitation, DNA, RNA, cDNA, gDNA, ssDNA, dsDNA or any combination thereof.

“Nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1 -yl (C) , guanine-9-yl (G), uracil- 1 -yl (U), and thymin-1 -yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “vector” or “construct” designates a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein “Interfering RNA” is any RNA involved in post-transcriptional gene silencing, which definition includes, but is not limited to, double stranded RNA (dsRNA), small interfering RNA (siRNA), and microRNA (miRNA) that are comprised of sense and antisense strands.

As used herein a “locked nucleic acid” (LNA), is a modified RNA nucleotide in which the ribose moiety is modified with an extra bridge connecting the 2′ oxygen and 4′ carbon. For example, a locked nucleic acid sequence comprises a nucleotide of the structure:

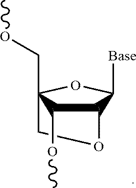

As used herein the term “neuropathy” defines a condition or disease involving dysfunction of one or more peripheral nerves, causing weakness, numbness, and pain from nerve damage, usually in the hands and feet. “Diabetic neuropathy” is defined as neuropathy that results in a subject with diabetes (either type I or type II).

As used herein the term “skin stroma cells” encompasses mesenchymal cells present in the dermis layer adjacent to the epidermis that release growth factors that promote cell division. For example skin stroma cells include fibroblasts and pericytes.

### Embodiments

Disclosed herein are method and compositions for treating neuropathy and stabilizing or stimulating the production of PGP9.5+ mature nerve fiber in a patient in need of neuroprotective measures. In one embodiment the patient is one who has Type I or Type II diabetes. More particularly, in one embodiment the present disclosure is directed to compositions and methods for reprogramming skin cells to exhibit one or more neurogenic properties relative to the the original unmodified cell. In one embodiment human dermal fibroblast cells are transfected in vivo by introducing into the human dermal fibroblast cells nucleic acid sequences that encoding for Ascl1, Brn2, and Myt1l. As disclosed herein such in vivo reprogramming of skin tissue cells results in changes in the associated tissue microenvironment that are benficial to adjacent peripheral nerves in a patient, and can be used for therapeutic purposes such as the rescue of pre-existing nerve fibers from their predictable path of loss in diabetic patients.

In one embodiment a method of treating neuropathy and/or reducing loss of cutaneous PGP9.5+ mature nerve fibers in a patient is provided. In one embodiment the method comprises the steps of reprogramming human dermal fibroblast cells in vivo by introducing into human dermal fibroblast cells nucleic acid sequences that encode Ascl1, Brn2, and Myt1l. In one embodiment the reprogrammed human dermal fibroblast cells exhibit one or more neurogenic properties relative to the original fibroblast cell, including at least one of the following properties:
enhanced Ngf expression;
enhanced expression of a neurotrophic factor gene selected from the group consising of Bdnf, Nt3, and Nt4/5; and
increased numbers of associated TuJ1+ cells associating with said reprogrammed cells in the dermis. In one embodiment the reprogrammed dermal fibroblasts continue to express fibroblast specific protein-1 (Fsp-1).

In one embodiment the present invention is directed to compositions comprising a neurogenic fibroblast that when present in skin tissue causes neurotrophic enrichment of the skin stroma, including elevation of endogenous NGF and other co-regulated neurotrophic factors such as Nt3, and methods of using such compositions to treat neuropathy and/or stabilizing or stimulating the production of PGP9.5+ mature nerve fiber in a patient in need of neuroprotective measures. The neurogenic fibroblasts disclosed herein are derived from validated human dermal fibroblasts (HADF) expressing fibroblast specific protein-1 (Fsp-1) that have been manipulated to have the capacity to promote a more neurogenic environment. Such reprogrammed fibroblasts are transcribed with nucleic acids that encode three gene products: Ascl1, Brn2, and Myt1l whose expression in skin fibroblasts converts the cells into electrophysiologically active induced neuronal cells (iN) and results in enrichment of the neurotrophic milieu of the skin. In one embodiment such reprogrammed fibroblasts are produced in vivo.

In accordance with one embodiment a method is provided for reprogramming human dermal fibroblast cells to exhibit one or more neurogenic properties relative to the original fibroblast cell, wherein the method comprises enhancing the activity and/or expression of Ascl1, Brn2, and Myt1l in said cells. In one embodiment the reprogrammed fibroblasts continue to express fibroblast specific protein-1 (Fsp-1), and exhibit at least of the following properties:
1. enhanced Ngf expression;
2. enhanced expression of a neurotrophic factor gene selected from the group consisting of Bdnf, Nt3, and Nt4/5; or
3. increased numbers of associated TuJ1+ cells associating with said reprogrammed cells in the dermis;

In accordance with one embodiment a method is provided for reprogramming human dermal fibroblast cells to exhibit one or more neurogenic properties relative to the original fibroblast cell, wherein the method comprises delivering intracellularly into the fibroblasts one or more proteins selected from the group consisting of Achaete-Scute Family BHLH Transcription Factor 1 (ASCL1), Master Neural Transcription Factor BRN2 (encoded by POU3F2), and Myelin Transcription Factor 1 Like (Myt1l), or polynucleotides encoding one or more proteins selected from the group consisting of ASCL1, BRN2, and Myt1l proteins; or exposing the fibroblasts to an extracellular vesicle produced from a cell containing or expressing one or more proteins selected from the group consisting of ASCL1, BRN2, and Myt1l, or polynucleotides encoding one or more proteins selected from the group consisting of ASCL1, BRN2, and Myt1l proteins.

In one embodiment the method of reprogramming the fibroblasts comprises increasing intracellular ASCL1, BRN2, and Myt1l protein concentrations, including for example by transfecting cells with one or more nucleic acid sequences that encode for ASCL1, BRN2, and Myt1l. The transfection can take place in vivo or in vitro. In one embodiment nucleic acid sequences encoding ASCL1, BRN2, and Myt1l are delivered into the cytosol of human dermal fibroblast cells in vivo. Any of the standard techniques for introducing macromolecules into cells can be used in accordance with the present invention. Known delivery methods can be broadly classified into two types. In the first type, a membrane-disruption-based method involving mechanical, thermal or electrical means can be used to disrupt the continuity of the cell membrane with enhanced permeabilization for direct penetration of desired macromolecules. In the second type, a carrier-based method, using various viruses, exosomes, vesicles and nanoparticle capsules, allows uptake of the carrier through endocytosis and fusion processes of cells for delivery of the carrier payload.

In one embodiment intracellular delivery is via a viral vector, or other delivery vehicle capable of interacting with a cell membrane to deliver its contents into a cell. In one embodiment intracellular delivery is via three-dimensional nanochannel electroporation, delivery by a tissue nanotransfection device, or delivery by a deep-topical tissue nanoelectroinjection device. In one embodiment the reprogramming composition is delivered into the cytosol of fibroblasts in vivo through tissue nanotransfection (TNT) using a silicon hollow needle array.

Among the methods of permeabilization-based disruption delivery, electroporation has already been established as a universal tool. High efficiency delivery can be achieved with minimum cell toxicity by careful control of the electric field distribution. In accordance with one embodiment nucleic acid sequences are delivered to the cytosol of somatic cells through the use of tissue nanotransfection (TNT). Tissue nanotransfection (TNT) is an electromotive gene transfer technology that delivers plasmids, RNA and oligonucleotides to live tissue causing direct conversion of tissue function in vivo under immune surveillance without the need for any laboratory procedures. Unlike viral gene transfer commonly used for in vivo tissue reprogramming, TNT obviates the need for a viral vector and thus minimizes the risk of genomic integration or cell transformation.

Current methods of in vivo reprogramming can involve transfecting cells in vivo or in vitro followed by implantation. Although one embodiment of the present invention entails in vitro reprogramming of cells followed by transplantation, cell implants are often met with low survival and poor tissue integration. Additionally, transfecting cells in vitro involves additional regulatory and laboratory hurdles.

In accordance with one embodiment demal fibroblasts are transfected in vivo with a reprogramming composition as disclosed herein. Common methods for bulk in vivo transfection are delivery of viral vectors or electroporation. Although viral vectors can be used in accordance with the present disclosure for delivery of a reprogramming composition to demal fibroblasts, viral vectors suffer the drawback of potentially initiating undesired immune reactions. In addition, many viral vectors cause long term expression of gene, which is useful for some applications of gene therapy, but for applications where sustained gene expression is unnecessary or even undesired, transient transfection is a viable option. Viral vectors also involve insertional mutagenesis and genomic integration that can have undesired side effects. However, in accordance with one embodiment certain non-viral carriers, such as liposomes or exosomes can be used to deliver a reprogramming cocktail to somatic cells in vivo.

TNT provides a method for localized gene delivery that causes direct conversion of tissue function in vivo under immune surveillance without the need for any laboratory procedures. By using TNT with plasmids, it is possible to temporally and spatially control overexpression of a gene or inhibit expression of a target gene. Spatial control with TNT allows for transfection of a target area such as a portion of skin tissue without transfection of other tissues. Details regarding TNT devices have been described in U.S. Published Pat. Application Nos. 20190329014 and 20200115425, the disclosures of which are expressly incorporated by reference. Tissue nanotransfection allows for direct cytosolic delivery of cargo (e.g , reprogramming factors) into cells by applying a highly intense and focused electric field through arrayed nanochannels, which benignly nanoporates the juxtaposing tissue cell members, and electrophoretically drives cargo into the cells.

In accordance with one embodiment a neurogenic fibroblast produced by any one of the methods disclosed herein is provided wherein the neurogenic fibroblast expresses fibroblast specific protein-1 (Fsp-1), and at least one protein selected from the group consisting of ASCL1, BRN2, and Myt1l. In one embodiment the neurogenic fibroblast is characterized by elevated expression of one or more of ASCL1, BRN2, and Myt1l, optionally by elevated expresion of all of ASCL1, BRN2, and Myt1l.

In accordance with the present invention the in vivo production of neurogenic fibroblasts disclosed herein can be used to stabilizing or stimulating production of PGP9.5+ mature nerve fiber in patients tissues, including a diabetic patient’s tissues. In one embodiment the method comprises the step of reprogramming dermal fibroblasts in vivo to become neurogenic, or introducing into the patient neurogenic fibroblasts that have been reprogrammed in vitro to be neurogenic. In one embodiment the demal fibroblasts have been reprogrammed by contacting said dermal fibroblasts with nucleic acid sequences encoding the proteins ASCL1, BRN2, and Myt1l under conditions that enhance cellular uptake of said nucleic acid sequences. In one embodiment the reprogramming comprises delivery of nucleic acid sequences encoding the proteins ASCL1, BRN2, and Myt1l into the cytosol of human dermal fibroblast cells via TNT.

In one embodiment a method of treating peripheral neuropathy in diabetic patients is provided wherein the method comprising introducing neurogenic fibroblasts or reprogramming fibroblasts to become neurogenic in tissues proximal to a site of neuropathic pain. In one embodiment the method comprises transfecting dermal fibroblast with nucleic acid sequences encoding the proteins ASCL1, BRN2, and Myt1l.

### Example 1

### Induction of a Neurogenic Fibroblast State

### Materials and Methods

### Mice

All animal studies were performed in accordance with protocols approved by the Institutional Laboratory Animal Care and Use Committee of Indiana University. Mice were maintained under standard conditions at 22 ± 2° C. with 12-h light/dark cycles and access to food and water ad libitum. C57B1/6 mice were purchased from Jackson laboratories. Lepr db/db mice homozygous (BKS.Cg7 m+/+Leprdb/J, or db/db; stock no 000642) for spontaneous mutation of the leptin receptor (Leprdb) (aged 8-10 weeks) were purchased from Jackson Laboratory, Bar Harbor, ME.

### Cell Culture

Primary Mouse Embryonic fibroblasts (MEFs) were purchased from Millipore Sigma (PMEF-HLC). MEFs were grown in DMEM supplemented with 10% fetal bovine serum, 100 ug/ml streptomycin, 100 U/ml penicillin, 0.25 ug/ml amphotericin and 1x MEM non-Essential amino acids (all from ThermoFisher Scientific). Cells were maintained at 37° C. in 95% air and 5% CO₂ in a humidified atmosphere.

### Nanochannel Electroporation for In Vitro Reprogramming

For nanochannel electroporation (NEP), cells were directly grown on the apical surface of a Transwell membrane (Corning cat#3460) at a density of ~0.15-0.18 × 10⁶ cells/well in regular maintenance medium (DMEM as mentioned above). The cells were allowed to adhere and spread overnight before nanochannel electroporation (NEP) transfection. Following cell loading, the media in the apical chamber was replaced by PBS and the Transwell inserts were then mounted on a custom made gold electrode in direct contact with the plasmid solution. A counter-electrode was then immersed in the PBS of the apical chamber, and a square wave pulse (275 V, 35 ms duration pulse, 1-10 pulses) was applied across the electrodes using a Biorad Gene Pulser Xcell power supply. The PBS was replaced by fresh media immediately after, and the cells were then incubated overnight at 37° C. Ascl1, Brn2, Myt1l (ABM) plasmids were mixed at a 2:1:1 molar ratio as described in D. Gallego-Perez et al, Nat Nanotechnol. (2017) 12:974-979.

### Induced Neuron Protocol

Post-NEP, MEF’s were cultured on Poly-D-lysine hydrobromide (Millipore Sigma, US) coated glass coverslips or plates in regular maintenance media for 24 h. After 24 h, media was replaced with neuronal induction medium. Neuronal induction media was prepared by supplementing DMEM base media with 1x N2 supplement, 100 ug/ml streptomycin, 100 U/ml penicillin, 0.25 ug/ml amphotericin, 1x MEM non-Eseential amino acids, and 10 ng/ml human bFGF. MEF cells transfected with ABM cDNA expression plasmids were differentiated for one, two or four weeks.

### Tissue Nanotransfection for In Vivo Reprogramming

For in vivo reprogramming, C57B1/6 mice (8-10 weeks old) or db/db mice (27-week-old) were used for tissue nano-transfection (TNT) to deliver nucleic acids sequences encoding for Ascl1, Brn2, and Myt11 (ABM) (TNT_{ABM} designating TNT conducted transfection of cells with ABM). The TNT device was used as described previously (D. Gallego-Perez, et al., Nat Nanotechnol. (2017);12:974-979). In brief, the dorsal area of skin to be used for transfection were depilated 24 h before TNT. The skin was then exfoliated to eliminate the dead/keratin cell layers to expose nucleated cells in epidermis. ABM plasmid cocktail (2:1:1 molar ratio) was loaded in the reservoir at a concentration of 0.05-0.1 ug/ul. A gold-coated electrode (cathode) was immersed in the plasmid solution, and a 25 G needle counter-electrode (anode) was inserted into the dermis juxtaposed to the TNT platform surface. Pulsed electrical stimulation (10 pulses, 250 V in amplitude, duration of 10 ms per pulse) was then applied across the electrodes to nanoporate the exposed cell membranes and drive the plasmid cargo into the cells through the nanochannels. Unless otherwise specified, control specimens involved TNT treatments with mock plasmid solution. After 24 h of TNT_{ABM}, mouse skin samples (12 mm punch biopsy) were collected in OCT. Histology of skin and mRNA expression in situ was performed on 10 µm-thick sections.

### DNA Plasmid Preparation

Mock (empty vector), Ascl1, Brn2, and Myt11 plasmids were prepared using a plasmid DNA purification kit (ZymoPURE II Plasmid Midiprep Kit,cat. no. D4201). DNA concentrations were obtained from Nanodrop 2000c Spectrophotemeter (Thermoscientific). Ascl1, Brn2, and Mytll plasmids (backbone, pCAGGs) were constructed with GFP (Ascl1), RFP (Brn2), or CFP (Myt1l) by Applied Biological Materials Inc., Richmond, BC, Canada) as previously described (D. Gallego-Perez et al, Nat Nanotechnol. (2017) 12:974-979). pCAGEN (empty) was a gift from Connie Cepko (Addgene plasmid#11160).

### RNA Isolation and Real-Time Quantitative PCR for mRNA

Total RNA was extracted by using the Total RNA Extraction and Purification Isolation Kit according to the manufacturer’s protocol (Norgen Biotek, Thorold, ON, Canada). For gene expression studies, total cDNA synthesis was achieved by using the SuperScript™ VILO™ cDNA Synthesis Kit (ThermoFisher Scientific). The abundance of mRNA for Ascl1, Brn2, Myt1l, Ngf, Bdnf, Nt3, Nt4/5 was quantified by real-time PCR by using SYBR Green-I. Gapdh served as housekeeping control. The following primer sets were used:

m_Gapdh_F:5′-ATGACCACAGTCCATGCCATCACT-3′(SEQ ID NO: 1)
m_Gapdh_R:5′-TGTTGAAGTCGCAGGAGACAACCT-3′(SEQ ID NO: 2)
m_Ascl1_F:5′-CGACGAGGGATCCTACGAC-3′(SEQ ID NO: 3)
m_Ascl1_R:5′-CTTCCTCTGCCCTCGAAC-3′(SEQ ID NO: 4)
m_Brn2_F:5′-GGTGGAGTTCAAGTCCATCTAC-3′(SEQ ID NO: 5)
m_Brn2_R:5′-TGGCGTCCACGTAGTAGTAG-3′(SEQ ID NO: 6)
m_Myt1l_F:5′-ATACAAGAGCTGTTCAGCTGTC-3′(SEQ ID NO: 7)
m_Myt1l_R:5′- GTCGTGCATATTTGCCACTG-3′(SEQ ID NO: 8)
m_Ngf_F:5′-ACCAATAGCTGCCCGAGTGACA-3′(SEQ ID NO: 9)
m_Ngf_R:5′-GAGAACTCCCCCATGTGGAAGACT-3′(SEQ ID NO: 10)
m_Bdnf_F:5′-CGTGGGGAGCTGAGCGTGTG-3′(SEQ ID NO: 11)
m_Bdnf_R:5′-GCCCCTGCAGCCTTCCTTGG-3′(SEQ ID NO: 12)
m_Nt3_F:5′-GCCCAAAGCAGAGGCACCCA-3′(SEQ ID NO: 13)
m_Nt3_R:5′-GCTACCACCGGGTTGCCCAC-3′(SEQ ID NO: 14)
m_Nt4/5_F:5′-AGTCTGCAGTCAACGCCCGC-3′(SEQ ID NO: 15)
m_Nt4/5_R:5′-TGCGACGCAGTGAGTGGCTG-3′(SEQ ID NO: 16)

### Immunocytochemistry (ICC)

ICC was performed on mouse embryonic fibroblasts (MEF) nano-transfected with neuronal conversion factors ABM, or mock plasmids. In brief, cells were fixed with 4% formaldehyde for 15 min at room temperature, permeabilized with 0.1 % Triton X-100 for 15 min followed by blocking in 10% normal goat serum for 1 h at room temperature. After blocking, primary antibody treatment was performed followed by three washing steps of PBS.

Secondary antibody was applied to visualize expression pattern of the MAP2 (Abcam, ab5392; 159 1:1000), beta III tubulin (TuJ1) (Abcam, ab52623; 1:200, GeneTex GTX85469; 1:500) and 160 Neurofilament 200 (Millipore Sigma N4142; 1: 200) proteins. The signal was visualized by subsequent incubation with appropriate fluorescence-tagged secondary antibodies (Alexa 488-tagged alpha-rabbit, 1:200; Alexa 568-tagged alpha-chicken, 1:200). Fluorescent images were acquired using the FluoView FV1000 spectral confocal microscope and laser scanning confocal microscope (LSM 880, Zeiss).

### Immunohistochemistry and Microscopy

Tissue immunostaining was carried out on 10 um thick paraffin or cryosections of 12 mm punch biopsy samples. Immunostainings of beta III tubulin (TuJ1) (Abcam, ab52623; 1:100; GeneTex, Inc. GTX85469, 1:500), S100A4 (Abcam, ab41532; 1:200), Nerve Growth Factor-beta (NFG) (Millipore Sigma, AB1526; 1:200), and Protein Gene Product 9.5 (PGP9.5) (Millipore Sigma, AB1761; 1:200), were performed on paraffin and cryosections of skin samples using specific antibodies as indicated. In brief, OCT or paraffin embedded tissue was cryosectioned at 10 um thick, fixed with cold acetone, blocked with 10% normal goat serum and incubated with specific antibodies. The signal was visualized by subsequent incubation with appropriate fluorescence-tagged secondary antibodies (Alexa 488 tagged alpha-rabbit, 1:200; Alexa 488 tagged alpha -chicken, 1:200; Alexa 568 tagged alpha-rabbit, 1:200) and counter-stained with DAPI. Images were collected using the Axio Scan.Z1 slide scanner (Zeiss Microscopy) or laser scanning confocal microscope (Zeiss). Image analysis software Zen (Zeiss) was used to quantitate fluorescence intensity. Additionally, a manual cell count of fluorescent positive cells in a field of view (FOV) using the cell count module in Zen (Zeiss). For each image, three-six such FOVs were counted and data represented as percent positive. Colocalization was performed using Zen black software.

### Enzyme-Linked Immunosorbent Assay (ELISA)

For cell culture experiments, NGF production was measured in culture media and normalized to total protein concentration measured from cell lysate. For skin tissue samples, protein was isolated from twenty 100 µM thick sections. Tissue sections were collected in HBSS, washed with HBSS 3x times to remove OCT and resuspended in homogenization buffer [50mM Tris-HCl pH7.5-8.0, 150 mM NaCl, 1% Triton X-100, 0.5% Sodium deoxycholate, 10 ul of protease inhibitor cocktail (Sigma, St. Louis, MO) and 10 ul of PMSF (100 mM)]. The tissue was homogenized on ice three times for 30 s each with 5- to 10-s breaks with Pellet Pestle Motor (Kimble Chase, NJ), followed by sonication on ice three times for 10 s each with 10-s breaks. The homogenate was centrifuged at 21.000 g for 5 min at 4° C. The supernatants were collected and stored at -80° C. until ELISA was performed. Bicinchoninic acid protein assay (Pierce, Rockford, IL) was performed according to the manufacturer’s instructions to standardize NGF values per milligram of protein. NGF protein levels were determined using NGF Rapid ELISA kit (Biosensis Pty Ltd).

### RNA In Situ Hybridization (Fluorescent Multiplex RNAscope)

Skin sections (10 um) were cut using a cryostat (Leica Microsystems) and mounted on Superfrost Plus Gold Glass Slides (Fisher Scientific, #22-035-813). Slides were subsequently stored at -80° C. Paired double-Z oligonucleotide probes were designed against target RNA using custom software. Probes against Ascl1 mRNA (313291-C2), Brn2 (460561-C3) and Myt1l (483401), as well as all other reagents for in-situ hybridization and DAPI labeling, were purchased from Advanced Cell Diagnostics (ACD, Newark, CA). The tissue pretreatment, hybridization, amplification, and detection were performed manually using RNAscope Multiplex Fluorescent Reagent v2 Kit according to manufacturer’s instructions. During RNAscope hybridization, positive probe (catalog #321811), negative probe (catalog #321831), and ABM probes were processed simultaneously. Fluorescent images were acquired using a FV3000 Olympus microscope.

### Results

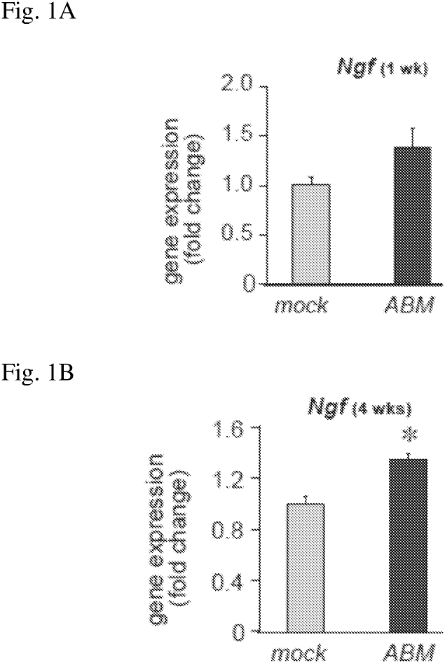

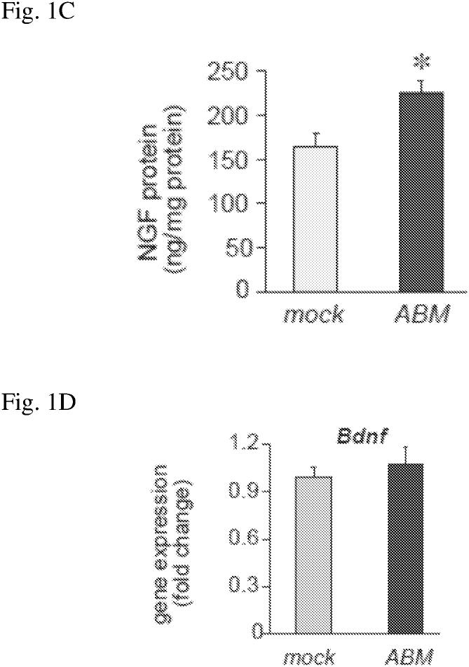

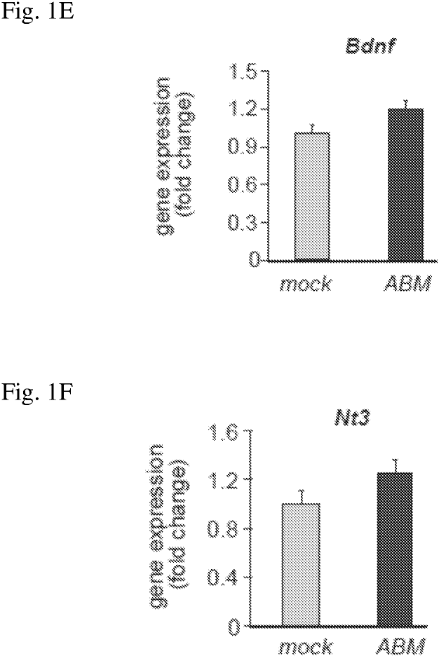

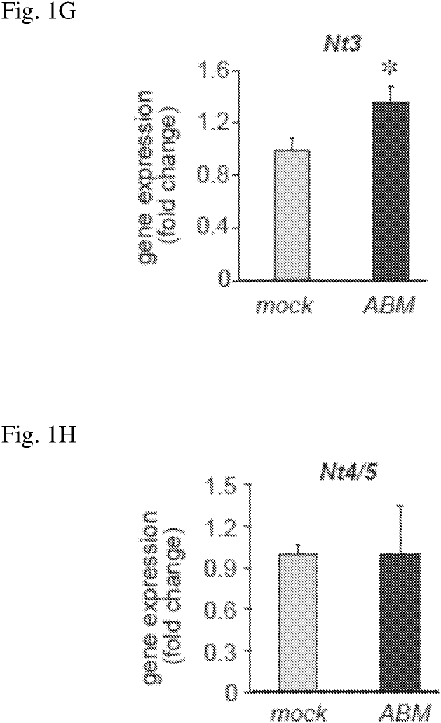

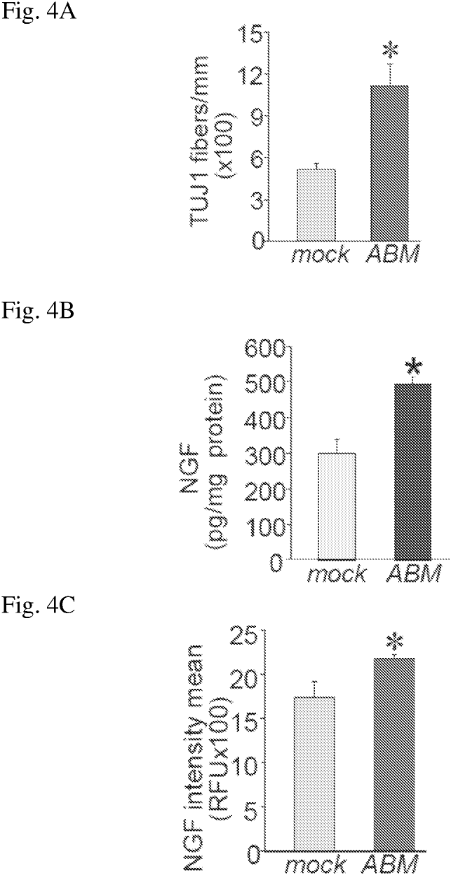

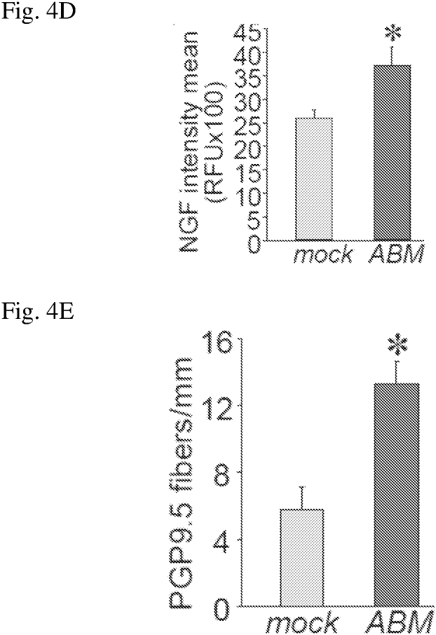

Delivery of ABM via nanochannel electroporation (NEPABM) led to conversion of MEF to iN cells 2 weeks and 4 weeks after transfection.. Induced neuronal (iN) cells, as indicated by neurofilament 200+ staining, showed significant elevated Ngf expression at 4 weeks, (FIG. 1B). NGF protein production was induced in MEF culture media at 4 weeks post-NEPABM as shown in FIG. 1C. Quantitative analysis of brain-derived neurotrophic factor (Bdnf) at 1 week (FIG. 1D) and 4 weeks (FIG. 1E) post-NEPABM, neurotrophin-3 (Nt3) at 1 week (FIG. 1F) and 4 weeks (FIG. 1G) post-NEPABM, and neurotrophin-⅘ (Nt4/5) at 1 week (FIG. 1H) and 4 weeks (FIG. 1I) post-NEPABM showed significant increase in the expression of Nt3 at 4 weeks post-NEPABM (FIG. 1G).

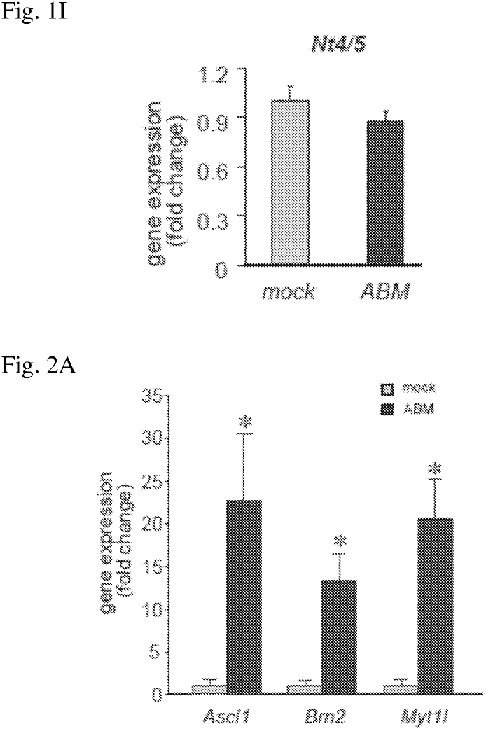

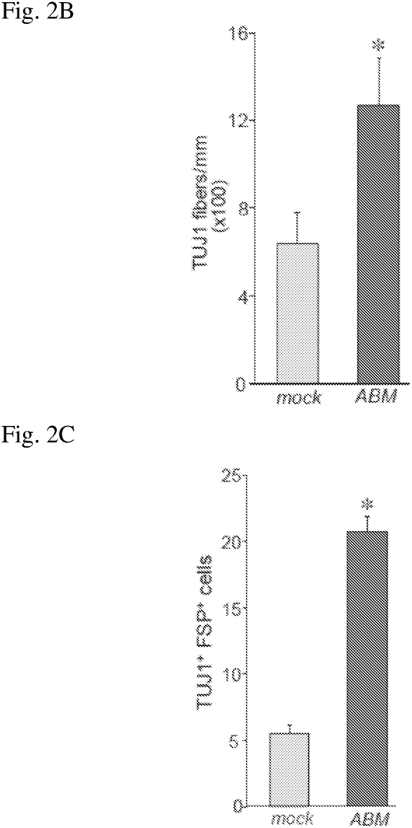

Successful topical delivery of ABM via TNT_{ABM} to the dorsal murine skin was validated in situ with detected expression of Ascl1, Brn2, and Myt1l (FIG. 2A). The iN cells, visualized in early phase as TuJ1+, were significantly abundant in the dermis at 4 weeks post-TNT_{ABM} (FIG. 2B). TuJ1+ iN cells co-expressed fibroblast-specific protein (FSP) marking that these iN cells were of fibroblasts origin. (FIG. 2C).

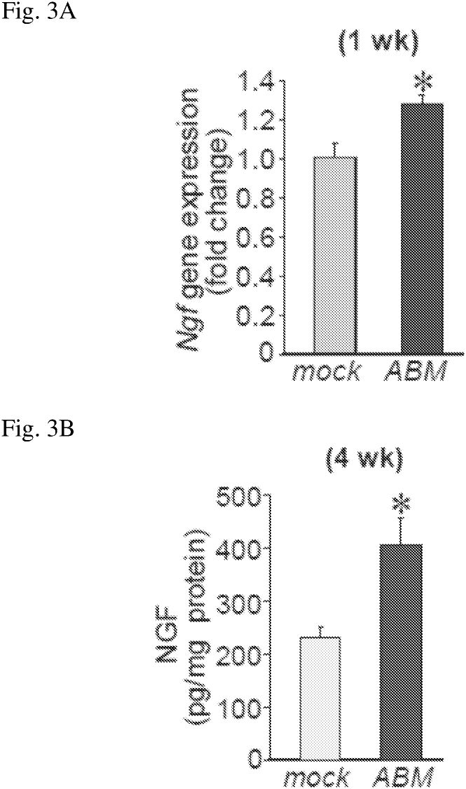

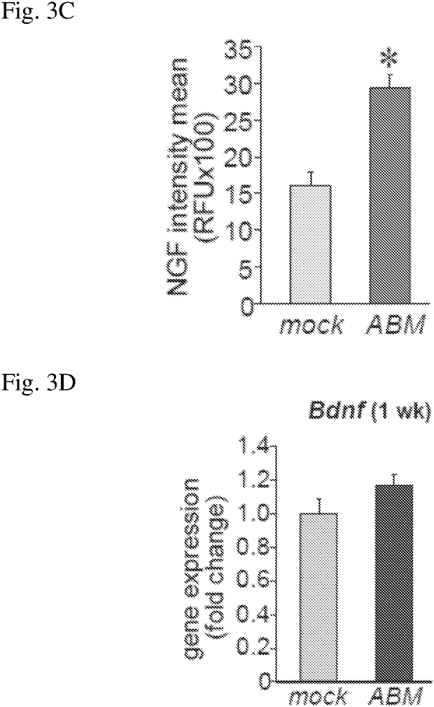

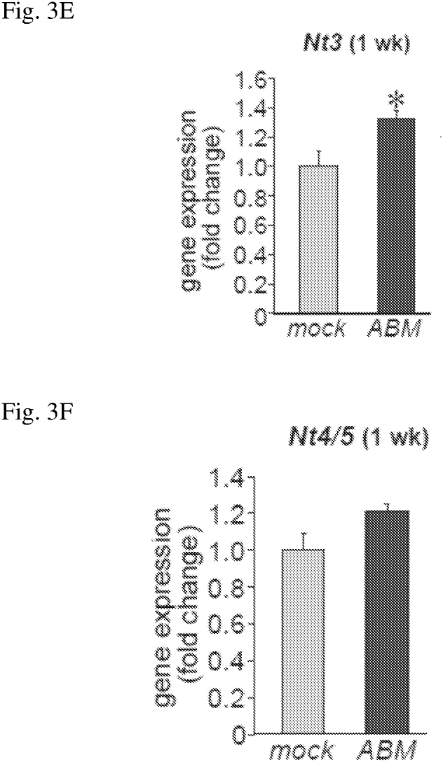

TNT_{ABM} enhanced Ngf expression in murine skin 1-week post-TNT_{ABM} (see FIG. 3A) followed by enhanced NGF protein production at 4 weeks post-TNT_{ABM} (FIG. 3B). Elevated NGF expression was localized in the epidermis based on immunostaining (FIG. 3C). Quantitative analysis of neurotrophic factor genes expression including Bdnf (FIG. 3D), Nt3 (FIG. 3E), and Nt4/5 (FIG. 3F) showed significant Nt3 expression at 1-week post-TNT_{ABM} (FIG. 3E).

Topical TNT_{ABM} on dorsal skin of db/db mice showed increased TuJ1+ cells in the dermis at 4 weeks (FIG. 4A). Abundance of NGF in the transfected tissue was quantified by ELISA (data is presented in FIG. 4B (n = 9.10), *p < 0.01) and quantification of the IHC images at 4 weeks (FIG. 4C) and at 9 weeks (FIG. 4D) showed significant increase of NGF at 4 weeks post-TNT_{ABM}. Elevated production of NGF by the epidermis was sustained for up to 9 weeks post-TNTABM in mice. These db/db mice were 36 weeks old at that time when the onset of neuropathy is well documented. Mature neurons as measured by PGP9.5+ staining was significantly higher in number compared to TNTmock (FIG. 4E).

### Discussion

In vivo reprogramming often relies on implantation of limited number of cells that have been reprogrammed in vitro. Such approach is often in conflict with the host immune system. Topical TNT mediated in vivo reprogramming offers the advantage that cells are converted within the live body under immune surveillance. Successful cell conversion in vivo, indicates that such reprogramming happened only after successful negotiation with the local immune system. Thus, such process of in vivo cell reprogramming is more likely to generate sustainable results with translational significance.

Reprogramming of cells in vivo induces the release of factors that are anticipated to affect non-reprogrammed cells within the same microenvironment by paracrine mechanisms. The products of in vivo reprogramming are successfully converted cells and a modified tissue microenvironment that is supportive of the survival and functionality of the converted cells and surrounding neurological cells. iN cells generated by TNT_{ABM} in the adult skin persist long-term and acquire electrophysiological activity.

As disclosed herein, TuJ1+ neural cells, produced in response to TNT_{ABM}, colocalized with FSP+ cells indicating fibroblast origin of iN as established previously. An interesting finding of this work is that the skin stroma enriches in NGF and Nt3 expression. Discrepant timeline of the induction of NGF and Nt3 under in vitro condition may be explained by differences in experimental conditions such as complexity of stroma and blood borne factors.

Delayed induction of NGF and NT3 expression was observed in aged diabetic mice indicative of barriers to successful neurogenic reprogramming under conditions of diabetes. Clinical assessment of DPN include sensory tests, nerve conduction velocity tests, or nerve fiber enumeration in skin biopsies by protein gene product 9.5 (PGP9.5) immunostaining. Enumeration of PGP9.5+ peptidergic and non-peptidergic intraepidermal nerve fibers (IENF) is increasingly recognized as the “gold standard” for quantitative assessment for small nerve loss in DPN. These early structural changes have been established in db/db mice. In this work, topical cutaneous TNT_{ABM} in db/db mice induced elevated NGF production for up to 9 weeks. Such elevated cutaneous NGF was associated with higher abundance of PGP9.5+ mature nerve fiber. It is well known that in db/db, cutaneous PGP9.5+ mature nerve fibers markedly diminished at this age. Thus, in response to topical cutaneous TNT_{ABM}, elevation of endogenous NGF and other co-regulated neurotrophic factors are effective in sparing loss of cutaneous PGP9.5+ mature nerve fibers in diabetes. Taken together, this is the first study demonstrating that under conditions of in vivo reprogramming, changes in the tissue microenvironment can be leveraged for therapeutic purposes such as the rescue of pre-existing nerve fibers from its predictable path of loss under conditions of diabetes.

1. A method of treating neuropathy in a patient, said method comprising
reprogramming human dermal fibroblast cells in vivo by introducing into human dermal fibroblast cells nucleic acid sequences that encoding for Ascl1, Brn2, and Myt1l, wherein said reprogrammed human dermal fibroblast cells exhibit one or more neurogenic properties relative to the original fibroblast cell, including at least of the following properties:
enhanced Ngf expression;
enhanced expression of a neurotrophic factor gene selected from the group consisting of Bdnf, Nt3, and Nt4/5; and
increased numbers of associated TuJ1+ cells associating with said reprogrammed cells in the dermis;
while said reprogrammed cells express fibroblast specific protein-1 (Fsp-1).

2. The method of claim 1 wherein said patient is diabetic.

3. The method of claim 1 wherein the intracellular delivery is via tissue nanotransfection.

4. A method of increasing the skin stroma cell expression of NGF and Nt3 expression in vivo, said method comprising the step of
introducing nucleic acid sequences that encode for Ascl1, Brn2, and Myt1l into said patient’s skin stroma cells via tissue nanotransfection.

5. A method of stabilizing or stimulating production of PGP9.5+ mature nerve fiber in a diabetic patient’s tissues, said method comprising the step of reprogramming dermal fibroblasts in vivo to become neurogenic, said method comprising
introducing nucleic acid sequences that encode for Ascl1, Brn2, and Myt1l into said patient’s dermal fibroblast cells, to produce reprogrammed dermal fibroblasts.

6. The method of claim 5 wherein said nucleic acid sequences are delivered into the cytosol of human dermal fibroblast cells via tissue nanotransfection.

7. The method of claim 5 wherein said method induces the morphofunctional restoration of peripheral nerves.


SEQUENCE LISTING (16 sequences)

SEQ ID NO: 1 - DNA, 24 bp, Artificial Sequence
  PCR Primer
atgaccacagtccatgccatcact

SEQ ID NO: 2 - DNA, 24 bp, Artificial Sequence
  PCR Primer
tgttgaagtcgcaggagacaacct

SEQ ID NO: 3 - DNA, 18 bp, Artificial Sequence
  PCR Primer
cgacgagggatcctacga

SEQ ID NO: 4 - DNA, 18 bp, Artificial Sequence
  PCR Primer
cttcctctgccctcgaac

SEQ ID NO: 5 - DNA, 21 bp, Artificial Sequence
  PCR Primer
ggtggagttcaagtccatcta

SEQ ID NO: 6 - DNA, 20 bp, Artificial Sequence
  PCR Primer
tggcgtccacgtagtagtag

SEQ ID NO: 7 - DNA, 22 bp, Artificial Sequence
  PCR Primer
atacaagagctgttcagctgtc

SEQ ID NO: 8 - DNA, 20 bp, Artificial Sequence
  PCR Primer
gtcgtgcatatttgccactg

SEQ ID NO: 9 - DNA, 22 bp, Artificial Sequence
  PCR Primer
accaatagctgcccgagtgaca

SEQ ID NO: 10 - DNA, 24 bp, Artificial Sequence
  PCR Primer
gagaactcccccatgtggaagact

SEQ ID NO: 11 - DNA, 20 bp, Artificial Sequence
  PCR Primer
cgtggggagctgagcgtgtg

SEQ ID NO: 12 - DNA, 20 bp, Artificial Sequence
  PCR Primer
gcccctgcagccttccttgg

SEQ ID NO: 13 - DNA, 20 bp, Artificial Sequence
  PCR Primer
gcccaaagcagaggcaccca

SEQ ID NO: 14 - DNA, 20 bp, Artificial Sequence
  PCR Primer
gctaccaccgggttgcccac

SEQ ID NO: 15 - DNA, 20 bp, Artificial Sequence
  PCR Primer
agtctgcagtcaacgcccgc

SEQ ID NO: 16 - DNA, 20 bp, Artificial Sequence
  PCR Primer
tgcgacgcagtgagtggctg